(19) United States
(12) Patent Application Publication
(10) Pub. No.: US 2023/0129723 A1
(43) Pub. Date: Apr. 27, 2023

(54) ENANTIOMERIC RATIOS OF 3,4-METHYLENEDIOXYMETHAMPHETAMINE AND RELATED METABOLITES AND USES THEREOF

(71) Applicant: ATAI Life Sciences AG, Berlin (DE)

(72) Inventors: Glenn SHORT, Scituate, MA (US); Carrie BOWEN, Uxbridge, MA (US)

(21) Appl. No.: 17/975,127

(22) Filed: Oct. 27, 2022

Related U.S. Application Data
(63) Continuation of application No. 17/958,251, filed on Sep. 30, 2022.
(60) Provisional application No. 63/250,978, filed on Sep. 30, 2021.

(51) Int. Cl. A61K 31/36; A61P 25/00; A61K 9/00

(52) U.S. Cl. CPC A61K 31/36; A61P 25/00; A61K 9/0053

(57) ABSTRACT

The present disclosure provides compositions comprising (R)-MDMA or a pharmaceutically acceptable salt thereof with no or substantially no (S)-MDMA, pharmaceutically acceptable salt thereof, or metabolite thereof. Also provided herein are methods of using the compositions for treating diseases/disorders, e.g., PTSD.

### CROSS-REFERENCE TO RELATED APPLICATION

The present application is a continuation of U.S. application Ser. No. 17/958,251, filed Sep. 30, 2022, which claims the benefit of priority to U.S. Provisional Application No. 63/250,978, filed Sep. 30, 2021, the contents of which are hereby incorporated by reference in its entirety.

### BACKGROUND OF THE INVENTION

### i. Field of the Invention

The present invention relates to enantiomeric ratios of 3,4-methylenedioxymethamphetamine and related metabolites and uses thereof.

### ii. Description of Related Art

MDMA (3,4-Methylenedioxymethamphetamine), also known as Ecstasy, is considered the prototype of a class of compounds called entactogens, which means “to touch within”, their main characteristic being their ability to increase feelings of love, empathy and closeness towards others. (Pitts, E. G., et al. Psychopharmacology 235, 377-392 (2017)). Structurally, MDMA is a ring-substituted phenethylamine with a chiral molecular center that gives rise to two stereoisomers: S(+)-MDMA and R(−)-MDMA; typically, effects of the former resemble those of psychostimulants and are primarily mediated by dopaminergic and noradrenergic pathways, including increases in motor activity and euphoria, whereas the latter induces qualitative effects similar to classical psychedelics, such as ego-dissolution and perceptive alterations, mediated by serotonergic pathways, including direct 5-HT2A receptor agonism. (Murnane, K. S., et al. J Pharmacol Exp Ther 331, 717-23 (2009)).

Racemic MDMA has demonstrated therapeutic benefit in treating certain conditions, including PTSD and generalized anxiety disorder, as well as other mental health disorders. However, in some cases, the administration of racemic MDMA has been associated with certain adverse events, including cardiac associated side effects. Therefore, there is a need for MDMA compositions that provide therapeutic benefit while reducing the side effects associated with administration of racemic MDMA.

### SUMMARY OF THE DISCLOSURE

In one aspect, the present disclosure provides pharmaceutical compositions including a non-racemic mixture of (R)-3,4-methylenedioxymethamphetamine (MDMA) or pharmaceutically acceptable salt thereof and (S)-MDMA or pharmaceutically acceptable salt thereof.

In embodiments, the administration of the compositions of the present disclosure to a patient in need thereof provides a greater therapeutic index compared to when an equal dose (by weight) of racemic MDMA is administered.

In embodiments, the weight ratio of (R)-MDMA to (S)-MDMA is greater than 1. In embodiments, the weight ratio of (R)-MDMA to (S)-MDMA is from 51:49 to 99:1. In embodiments, the weight ratio of (R)-MDMA to (S)-MDMA is from 90:10 to 99:1. In embodiments, the weight ratio of (R)-MDMA to (S)-MDMA is greater than 90:10.

In embodiments, the weight ratio of (R)-MDMA to (S)-MDMA is less than 1.

In one aspect, the present disclosure provides pharmaceutical composition including a non-racemic mixture of a (R)-MDMA metabolite or pharmaceutically acceptable salt thereof and a (S)-MDMA metabolite or pharmaceutically acceptable salt thereof.

In some embodiments, the MDMA metabolite is 3,4-methylenedioxyamphetamine (MDA), 4-hydroxy-3-methoxymethamphetamine (HMMA), 4-hydroxy-3-methoxyamphetamine (HMA), 3,4-dihydroxyamphetamine (DHA), 3,4-methylenedioxyphenylacetone (MDP2P) or 3,4-methylenedioxy-N-hydroxyamphetamine.

In embodiments, the MDMA metabolite is MDA. In embodiments, the weight ratio of (R)-MDA to (S)-MDA is greater than 1. In embodiments, the weight ratio of (R)-MDA to (S)-MDA is from 90:10 to 99:1. In embodiments, the weight ratio of (R)-MDA to (S)-MDA is greater than 90:10.

In embodiments, the composition of the present disclosure is an oral dosage form. In embodiments, the oral dosage form is a tablet or capsule.

In one aspect, the present disclosure provides methods of reducing the incidence of adverse events associated with the administration of racemic MDMA by administering a therapeutically effective amount of the composition of the present disclosure.

In embodiments, the adverse event associated with the administration of racemic MDMA is hypertension or stroke.

In embodiments, the patient administered a composition of the present disclosure is treated for symptoms of post-traumatic stress disorder (PTSD). In embodiments, the patient administered a composition of the present disclosure is treated for symptoms of generalized anxiety disorder. In embodiments, the patient administered a composition of the present disclosure is treated for an eating disorder.

In embodiments, the administered ratio of (R)-MDMA to (S)-MDMA, or a metabolite thereof, and dose of the composition of the present disclosure is selected to reduce the incidence of adverse events in obese patients.

In embodiments, the administered ratio of (R)-MDMA to (S)-MDMA, or a metabolite thereof, and dose of the composition of the present disclosure is selected to reduce the incidence of adverse events in hypertensive patients.

### BRIEF DESCRIPTION OF THE DRAWINGS

FIG. 1 shows the effects of Racemic R,S(+/−)-MDMA on neurite outgrowth measures in rat embryonic cortical neuron cultures.

FIG. 2 shows the effects of R(−)-MDMA on neurite outgrowth measures in rat embryonic cortical neuron cultures.

FIG. 3 shows the effects of S(+)-MDMA on neurite outgrowth measures in rat embryonic cortical neuron cultures.

FIG. 4 shows the effects of Racemic R, S(+/−)-MDA on neurite outgrowth measures in rat embryonic cortical neuron cultures.

FIG. 5 shows the effects of R(−)-MDA on neurite outgrowth measures in rat embryonic cortical neuron cultures.

FIG. 6 shows the effects of S(+)-MDA on neurite outgrowth measures in rat embryonic cortical neuron cultures.

FIG. 7 shows the effects of racemic and non-racemic MDMA enantiomeric ratios R(−)≥S(+) on neurite outgrowth in rat embryonic cortical neuron cultures.

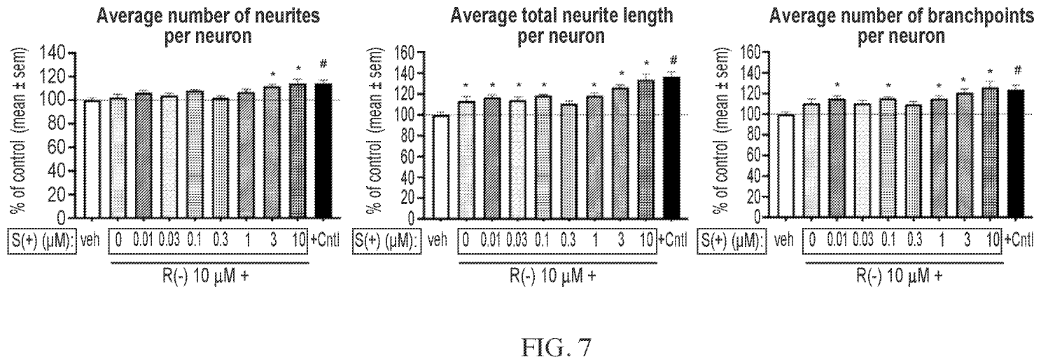

FIG. 8 shows the effects of racemic and non-racemic MDMA enantiomeric ratios of S(+)≥R(−) on neurite outgrowth in rat embryonic cortical neuron cultures.

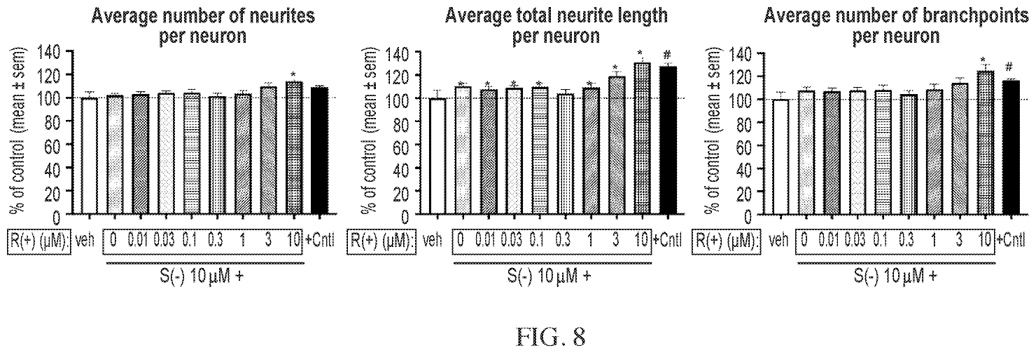

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55,“about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt thereof, that, when administered to a patient, is capable of performing the intended result. For example, an effective amount of the composition of the present disclosure is that amount that is required to reduce at least one symptom of patient's condition (for example, post-traumatic stress disorder (PTSD), generalized anxiety disorder, or eating disorder). The actual amount that comprises the “effective amount” or “therapeutically effective amount” will vary depending on a number of conditions including, but not limited to, the severity of the disorder, the size and health of the patient, and the route of administration.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein with regard to a patient, refers to improving at least one symptom of the patient's headache (for example, headache). Treating can be improving, or at least partially ameliorating a disorder.

As used herein, “therapeutically-effective dose” means a dose sufficient to achieve the intended therapeutic purpose, such as, to alleviate a sign or symptom of a disease or disorder in a patient. The “doses” described herein are expressed in terms of the active moiety (e.g., (R)-MDMA free base) rather than salt strength equivalent (e.g., (R)-MDMA hydrochloride). For example, doses of (R)-MDMA described herein are expressed in terms of the amount of MDMA free base (Formula Weight=193.24 g/mol) that is present in a composition of the present disclosure or administered according to a method of the present disclosure.

The term “therapeutic effect” as used herein refers to a desired or beneficial effect provided by the method and/or the composition. For example, the method for treating PTSD provides a therapeutic effect when the method reduces at least one symptom of PTSD in a patient.

### DETAILED DESCRIPTION OF PREFERRED EMBODIMENTS

The compositions of the present disclosure include (R)-MDMA and (S)-MDMA in various ratios to provide certain beneficial aspects while limiting known side effects of the racemate. (Kilpatrick, D. G. et al. J Trauma Stress 26, 537-47 (2013)). Thus, in embodiments, the compositions of the present disclosure produce specific pharmacological profiles and resulting changes in neurochemistry to support tailoring a specific ratio for a specific mental health indication.

### Compositions of the Disclosure:

The present disclosure provides pharmaceutical compositions containing a non-racemic mixture of (R)-3,4-methylenedioxymethamphetamine (MDMA) or pharmaceutically acceptable salt thereof and (S)-MDMA or pharmaceutically acceptable salt thereof.

In embodiments, the administration of a composition of the present disclosure to a patient in need thereof provides a greater therapeutic index compared to when an equal dose (by weight) of racemic MDMA is administered.

In embodiments, the administration of a composition of the present disclosure to a patient in need thereof provides increased levels of neurohormones (such as oxytocin, vasopressin and prolactin) compared to when an equal dose (by weight) of substantially pure (R)-MDMA is administered.

In embodiments, the administration of a composition of the present disclosure to a patient in need thereof provides increased stimulation of post-synaptic 5-HT2A signaling compared to when an equal dose (by weight) of racemic MDMA is administered.

In embodiments, the administration of a composition of the present disclosure to a patient in need thereof provides increased neurogenesis compared to when an equal dose (by weight) of racemic MDMA is administered.

In embodiments, the present disclosure provides pharmaceutical compositions comprising a non-racemic mixture of (R)-3,4-methylenedioxymethamphetamine (MDMA) or pharmaceutically acceptable salt thereof and (S)-MDMA or pharmaceutically acceptable salt thereof.

In embodiments, the weight ratio of (R)-MDMA to (S)-MDMA is greater than 1. In embodiments, the weight ratio of (R)-MDMA to (S)-MDMA is greater than 2, 3, 4 or 5. In embodiments, the weight ratio of (R)-MDMA to (S)-MDMA is greater than 5.

In embodiments, the weight ratio of (R)-MDMA to (S)-MDMA is from 51:49 to 99:1. In embodiments, the weight ratio of (R)-MDMA to (S)-MDMA is selected from the group consisting of 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 and 99:1.

In embodiments, the weight ratio of (R)-MDMA to (S)-MDMA is from 90:10 to 99:1.

In embodiments, the weight ratio of (R)-MDMA to (S)-MDMA is selected from the group consisting of 90:10, 91:9, 92:8, 93:7, 94:6, 95:5, 96:4, 97:3, 98:2 and 99:1.

In embodiments, the weight ratio of (R)-MDMA to (S)-MDMA is greater than 90:10. In embodiments, the weight ratio of (R)-MDMA to (S)-MDMA is greater than 95:5, 96:4, 97:3, 98:2 or 99:1. In embodiments, the weight ratio of (R)-MDMA to (S)-MDMA is greater than 95:5.

In embodiments, the weight ratio of (R)-MDMA to (S)-MDMA is less than 1.

In embodiments, the present disclosure provides a pharmaceutical composition comprising a non-racemic mixture of a (R)-MDMA metabolite and a (S)-MDMA metabolite, or pharmaceutically acceptable salt thereof.

In embodiments, the MDMA metabolite is selected from the group consisting of 3,4-methylenedioxyamphetamine (MDA), 4-hydroxy-3-methoxymethamphetamine (HMMA), 4-hydroxy-3-methoxyamphetamine (HMA), 3,4-dihydroxyamphetamine (DHA), 3,4-methylenedioxyphenylacetone (MDP2P) and 3,4-methylenedioxy-N-hydroxyamphetamine.

In embodiments, the MDMA metabolite is MDA.

In embodiments, the administration of a composition of the present disclosure to a patient in need thereof provides a greater therapeutic index compared to when an equal dose (by weight) of a racemic MDMA metabolite is administered.

In embodiments, the administration of a composition of the present disclosure to a patient in need thereof provides increased stimulation of post-synaptic 5-HT2A signaling compared to when an equal dose (by weight) of a racemic MDMA metabolite is administered.

In embodiments, the administration of a composition of the present disclosure to a patient in need thereof provides increased neurogenesis compared to when an equal dose (by weight) of a racemic MDMA metabolite is administered.

In embodiments, the weight ratio of a (R)-MDMA metabolite to a (S)-MDMA metabolite is greater than 1. In embodiments, the weight ratio of a (R)-MDMA metabolite to a (S)-MDMA metabolite is greater than 2, 3, 4 or 5. In embodiments, the weight ratio of a (R)-MDMA metabolite to a (S)-MDMA metabolite is greater than 5.

In embodiments, the weight ratio of a (R)-MDMA metabolite to a (S)-MDMA metabolite is from 51:49 to 99:1. In embodiments, the weight ratio of a (R)-MDMA metabolite to a (S)-MDMA metabolite is selected from the group consisting of 51:49, 52:48, 53:47, 54:46, 55:45, 56:44, 57:43, 58:42, 59:41, 60:40, 61:39, 62:38, 63:37, 64:36 , 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 and 99:1.

In embodiments, the weight ratio of a (R)-MDMA metabolite to a (S)-MDMA metabolite is from 90:10 to 99:1. In embodiments, the weight ratio of a (R)-MDMA metabolite to a (S)-MDMA metabolite is selected from the group consisting of 90:10, 91:9, 92:8, 93:7, 94:6, 95:5, 96:4, 97:3, 98:2 and 99:1.

In embodiments, the weight ratio of a (R)-MDMA metabolite to a (S)-MDMA metabolite is greater than 90:10. In embodiments, the weight ratio of a (R)-MDMA metabolite to a (S)-MDMA metabolite is greater than 95:5, 96:4, 97:3, 98:2 or 99:1. In embodiments, the weight ratio of a (R)-MDMA metabolite to a (S)-MDMA metabolite is greater than 95:5.

In embodiments, the present disclosure provides a pharmaceutical composition comprising a mixture of a (R)-MDMA or a pharmaceutically acceptable salt thereof and a (S)-MDMA metabolite or pharmaceutically acceptable salt thereof. In embodiments, the MDMA metabolite is (S)-MDA

In embodiments, the weight ratio of (R)-MDMA to (S)-MDA is greater than 1. In embodiments, the weight ratio of (R)-MDMA to (S)-MDA is greater than 2, 3, 4 or 5. In embodiments, the weight ratio of (R)-MDMA to (S)-MDA is greater than 5.

In embodiments, the weight ratio of (R)-MDMA to (S)-MDA is from 51:49 to 99:1. In embodiments, the weight ratio of (R)-MDMA to (S)-MDA is selected from the group consisting of 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 and 99:1.

In embodiments, the weight ratio of (R)-MDMA to (S)-MDA is from 90:10 to 99:1.

In embodiments, the weight ratio of (R)-MDMA to (S)-MDA is selected from the group consisting of 90:10, 91:9, 92:8, 93:7, 94:6, 95:5, 96:4, 97:3, 98:2 and 99:1.

In embodiments, the weight ratio of (R)-MDMA to (S)-MDA is greater than 90:10. In embodiments, the weight ratio of (R)-MDMA to (S)-MDA is greater than 95:5, 96:4, 97:3, 98:2 or 99:1. In embodiments, the weight ratio of (R)-MDMA to (S)-MDA is greater than 95:5.

In embodiments, the present disclosure provides MDMA wherein the MDMA comprises (R)-MDMA that is substantially free of (S)-MDMA, metabolite thereof (e.g., S-MDA), or a pharmaceutically acceptable salt thereof.

In embodiments, the present disclosure provides (R)-MDMA in an enantiomeric purity of ≥90%, ≥95%, ≥96%, ≥97%, ≥98%, ≥99%, ≥99.5%, ≥99.9%, or ≥99.99%. In embodiments, the present disclosure provides (R)-MDMA in an enantiomeric purity of ≥98%. In embodiments, the present disclosure provides (R)-MDMA in an enantiomeric purity of 100%.

In embodiments, the present disclosure provides pharmaceutical compositions containing MDMA wherein the compositions comprise (R)-MDMA that is substantially free of (S)-MDMA, metabolite thereof (e.g., S-MDA), or a pharmaceutically acceptable salt thereof.

In embodiments, the present disclosure provides a pharmaceutical composition containing MDMA, wherein the composition contains (R)-MDMA and less than 10%, less than 5%, less than 1%, less than 0.01%, less than 0.001%, less than 0.0001%, or less than 0.00001% (by weight) of (S)-MDMA, metabolite thereof (e.g., S-MDA), or a pharmaceutically acceptable salt thereof.

In embodiments, the compositions of the present disclosure comprise (R)-MDMA and no or substantially no (S)-MDMA, metabolite thereof (e.g., S-MDA), or a pharmaceutically acceptable salt thereof.

In embodiments, the enantiomeric purity of the (R)-MDMA in the pharmaceutical compositions of the present disclosure is ≥90%, ≥95%, ≥96%, ≥97%, ≥98%, ≥99%, ≥99.5%, ≥99.9%, or ≥99.99%. In embodiments, enantiomeric purity of the (R)-MDMA in the pharmaceutical compositions of the present disclosure is ≥98%. In embodiments, enantiomeric purity of the (R)-MDMA in the pharmaceutical compositions of the present disclosure is 100%.

In embodiments, the (R)-MDMA, (S)-MDA, or metabolite thereof is a hydrochloride salt of (R)-MDMA, (S)-MDA, or metabolite thereof.

In embodiments, the (R)-MDMA is a hydrochloride salt.

The compositions of the present disclosure may be prepared using methods that are known to those skilled in the art. For example, stereochemically pure R(−)-MDMA (and stereochemically pure S(+)-MDMA) may be synthesized and the purified stereoisomers combined in the ratios described herein. Racemic MDMA may also be resolved using chiral salt purification methods to provide stereochemically pure (R)-MDMA and (S)-MDMA. Similar methods may be used to provide the presently disclosed mixtures of (R)-MDMA and (S)-MDMA metabolites, such as MDA.

The compositions of the present disclosure are formulated for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, and oral solutions or suspensions, and oil-water emulsions containing suitable quantities of the MDMA composition described herein.

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art.

In embodiments, the disclosure provides a pharmaceutical composition comprising a non-racemic mixture of (R)-MDMA or pharmaceutically acceptable salt thereof and (S)-MDMA or pharmaceutically acceptable salt thereof and one or more pharmaceutically acceptable carriers or excipients.

In embodiments, provided herein are oral dosage forms comprising (i) an (R)-MDMA enantiomer comprising (R)-MDMA or pharmaceutically acceptable salt thereof; and (ii) a pharmaceutically acceptable carrier. In embodiments, the enantiomeric purity of the (R)-MDMA in the oral dosage form of the present disclosure is ≥90%, ≥95%, ≥96%, ≥97%, ≥98%, ≥99%, ≥99.5%, ≥99.9%, or ≥99.99%. In embodiments, enantiomeric purity of the (R)-MDMA in the oral dosage form of the present disclosure is ≥98%. In embodiments, enantiomeric purity of the (R)-MDMA in the oral dosage form of the present disclosure is 100%.

In embodiments, the oral dosage form comprises a hydrochloride salt of (R)-MDMA.

In embodiments, the oral dosage form comprises about 75 mg to about 1000 mg of (R)-MDMA, about 225 mg to about 600 mg of (R)-MDMA, about 500 mg to about 600 mg of (R)-MDMA, or about 300 mg to about 600 mg, based on (R)-MDMA free base. In embodiments, the oral dosage form comprises about 75 mg, about 85 mg, about 95 mg, about 105 mg, about 110 mg, about 115 mg, about 125 mg, about 135 mg, about 145 mg, about 155 mg, about 165 mg, about 175 mg, about 185 mg, about 195 mg, about 205 mg, about 215 mg, about 225 mg, about 235 mg, about 245 mg, about 255 mg, about 265 mg, about 275 mg, about 285 mg, about 295 mg, about 305 mg, about 315 mg, about 325 mg, about 335 mg, about 345 mg, about 355 mg, about 365 mg, about 375 mg, about 385 mg, about 395 mg, about 405 mg, about 415 mg, about 425 mg, about 435 mg, about 445 mg, about 455 mg, about 465 mg, about 475 mg, about 485 mg, about 495 mg, about 505 mg, about 515 mg, about 525 mg, about 535 mg, about 545 mg, about 555 mg, about 565 mg, about 575 mg, about 585 mg, about 595 mg, about 605 mg, about 615 mg, about 625 mg, about 635 mg, about 645 mg, about 655 mg, about 665 mg, about 675 mg, about 685 mg, about 695 mg, about 705 mg, about 715 mg, about 725 mg, about 735 mg, about 745 mg, about 755 mg, about 765 mg, about 775 mg, about 785 mg, about 795 mg, about 805 mg, about 815 mg, about 825 mg, about 835 mg, about 845 mg, about 855 mg, about 865 mg, about 875 mg, about 885 mg, about 895 mg, about 905 mg, about 915 mg, about 925 mg, about 935 mg, about 945 mg, about 955 mg, about 965 mg, about 975 mg, about 985 mg, about 995 mg, or about 1000 mg, including all values and ranges therebetween.

In embodiments, the oral dosage form comprises about 40 mg to 220 mg of (R)-MDMA or pharmaceutically acceptable salt thereof. In embodiments, the oral dosage form comprises about 20 mg, about 25 mg, about 30 mg, about 35 mg, about 40 mg, about 45 mg, about 50 mg, about 55mg, about 60 mg, about 65mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg or about 180 mg of (R)-MDMA or pharmaceutically acceptable salt thereof, including all values and ranges there between.

In embodiments, an oral dosage form described herein comprises (R)-MDMA, wherein the enantiomeric excess of the (R)-MDMA is ≥90%, ≥95%, ≥96%, ≥97%, ≥98%, ≥99%, ≥99.9%, or ≥99.99%. In embodiments, enantiomeric purity of the (R)-MDMA in the oral dosage form of the present disclosure is ≥98%. In embodiments, enantiomeric purity of the (R)-MDMA in the oral dosage form of the present disclosure is 100%.

In embodiments, provided herein are oral dosage forms comprising (i) an (R)-MDMA enantiomer comprising greater than 98% of (R)-MDMA or pharmaceutically acceptable salt thereof; and (ii) a pharmaceutically acceptable carrier.

In embodiments, provided herein are oral dosage forms comprising (i) about 225 to about 1000 mg, based on free base amount, of a hydrochloride (HCl) salt of an (R)-MDMA enantiomer comprising greater than 98% (R)-MDMA; and (ii) a pharmaceutically acceptable carrier.

### Methods of the Disclosure:

In one aspect, the present disclosure provides methods of administering the compositions of the present disclosure to a patient in need thereof. In embodiments, the methods of the present disclosure comprise administering a therapeutically effective amount of a non-racemic mixture of (R)-MDMA or pharmaceutically acceptable salt thereof or (S)-MDMA or pharmaceutically acceptable salt thereof to a patient in need thereof.

In embodiments, the present disclosure provides a method of reducing the incidence of adverse events associated with the administration of racemic MDMA, the method comprising administering a therapeutically effective amount of a composition of the present disclosure to a patient in need thereof.

In embodiments, the adverse event associated with the administration of racemic MDMA is hypertension. In embodiments, the adverse event associated with the administration of racemic MDMA is stroke.

In embodiments, the present disclosure provides methods of treating generalized anxiety disorder by administering a therapeutically effective amount of a composition of the present disclosure to a patient in need thereof.

In embodiments, the present disclosure provides methods of treating an eating disorder by administering a therapeutically effective amount of composition of the present disclosure to a patient in need thereof.

In embodiments, the administered ratio of (R)-MDMA to (S)-MDMA, or a metabolite thereof, and dose of the composition is selected to reduce the incidence of adverse events in obese patients.

In embodiments, the administered ratio of (R)-MDMA to (S)-MDMA, or a metabolite thereof, and dose of the composition is selected to reduce the incidence of adverse events in hypertensive patients.

In embodiments, the present disclosure provides methods of treating post-traumatic stress disorder (PTSD) by administering a therapeutically effective amount of a composition of the present disclosure to a patient in need thereof.

In embodiments, the present disclosure provides a method of treating PTSD in a patient in need thereof, the method comprising administering a non-racemic mixture of (R)-MDMA or pharmaceutically acceptable salt thereof, (S)-MDMA or pharmaceutically acceptable salt thereof, or a pharmaceutical composition described herein to the patient in need thereof.

In embodiments, the present disclosure provides a method of treating PTSD in a patient in need thereof, the methods comprising administering (R)-MDMA, or a pharmaceutically acceptable salt thereof or a pharmaceutical composition comprising the same to the patient, wherein the composition comprises substantially no (S)-MDMA or metabolite thereof.

In embodiments, the present disclosure provides a method of treating PTSD in a patient in need thereof, the method comprising administering to the patient an effective amount of an oral dosage form comprising: (i) an (R)-MDMA enantiomer comprising greater than 98% of (R)-MDMA or pharmaceutically acceptable salt thereof; and (ii) a pharmaceutically acceptable carrier. In embodiments, the enantiomeric purity of the (R)-MDMA in the administered oral dosage form is ≥90%, ≥95%, ≥96%, ≥97%, ≥98%, ≥99%, ≥99.5%, ≥99.9%, or ≥99.99%. In embodiments, enantiomeric purity of the (R)-MDMA in the administered oral dosage form is ≥98%. In embodiments, enantiomeric purity of the (R)-MDMA in the administered oral dosage form is 100%. In embodiments, (R)-MDMA hydrochloride (HCl) salt is administered.

In embodiments, the administered oral dosage form comprises about 75 mg to about 1000 mg (R)-MDMA, based on (R)-MDMA free base. In embodiments, the administered oral dosage form comprises about 225 mg to about 600 mg (R)-MDMA. In embodiments, the administered oral dosage from comprises about 500 mg to about 600 mg (R)-MDMA. In embodiments, the administered oral dosage from comprises about 300 mg to about 600 mg (R)-MDMA.

In embodiments, the present disclosure provides a method of treating PTSD in a patient in need thereof, the method comprising administering to the patient an oral dosage form comprising: (i) about 225 mg to about 1000 mg, based on free base amount, of a hydrochloride (HCl) salt of an (R)-MDMA enantiomer comprising greater than 98% of (R)-MDMA or pharmaceutically acceptable salt thereof; and (ii) a pharmaceutically acceptable carrier. In embodiments, the administered oral dosage form comprises greater than about 99% of (R)-MDMA. In embodiments, the administered oral dosage form comprises 100% of (R)-MDMA. In embodiments, the administered oral dosage form comprises about 500 mg to about 600 mg of (R)-MDMA. In embodiments, the administered oral dosage form comprises about 300 mg to about 600 mg of (R)-MDMA.

In embodiments, the present disclosure provides a method of treating a condition or disorder in a patient in need thereof (such as PTSD), the method comprising administering to the patient about 20 mg to 220 mg of a non-racemic mixture of (R)-MDMA or pharmaceutically acceptable salt thereof or (S)-MDMA or pharmaceutically acceptable salt thereof. In embodiments, the present disclosure provides a method of treating a condition or disorder in a patient in need thereof (such as PTSD), the method comprising administering to the patient about 20 mg, about 25 mg, about 30 mg, about 35 mg, about 40 mg, about 45 mg, about 50 mg, about 55mg, about 60 mg, about 65mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg or about 180 mg of a non-racemic mixture of (R)-MDMA or pharmaceutically acceptable salt thereof or (S)-MDMA or pharmaceutically acceptable salt thereof, including all values and ranges there between.

In embodiments, the present disclosure provides a method of treating a condition or disorder in a patient in need thereof (such as PTSD), the method comprising administering to the patient about 75 mg to 1000 mg of a non-racemic mixture of (R)-MDMA or pharmaceutically acceptable salt thereof or (S)-MDMA or pharmaceutically acceptable salt thereof. In embodiments, the present disclosure provides a method of treating a condition or disorder in a patient in need thereof (such as PTSD), the method comprising administering to the patient about 75 mg, about 85 mg, about 95 mg, about 105 mg, about 110 mg, about 115 mg, about 125 mg, about 135 mg, about 145 mg, about 155 mg, about 165 mg, about 175 mg, about 185 mg, about 195 mg, about 205 mg, about 215 mg, about 225 mg, about 235 mg, about 245 mg, about 255 mg, about 265 mg, about 275 mg, about 285 mg, about 295 mg, about 305 mg, about 315 mg, about 325 mg, about 335 mg, about 345 mg, about 355 mg, about 365 mg, about 375 mg, about 385 mg, about 395 mg, about 405 mg, about 415 mg, about 425 mg, about 435 mg, about 445 mg, about 455 mg, about 465 mg, about 475 mg, about 485 mg, about 495 mg, about 505 mg, about 515 mg, about 525 mg, about 535 mg, about 545 mg, about 555 mg, about 565 mg, about 575 mg, about 585 mg, about 595 mg, about 605 mg, about 615 mg, about 625 mg, about 635 mg, about 645 mg, about 655 mg, about 665 mg, about 675 mg, about 685 mg, about 695 mg, about 705 mg, about 715 mg, about 725 mg, about 735 mg, about 745 mg, about 755 mg, about 765 mg, about 775 mg, about 785 mg, about 795 mg, about 805 mg, about 815 mg, about 825 mg, about 835 mg, about 845 mg, about 855 mg, about 865 mg, about 875 mg, about 885 mg, about 895 mg, about 905 mg, about 915 mg, about 925 mg, about 935 mg, about 945 mg, about 955 mg, about 965 mg, about 975 mg, about 985 mg, about 995 mg, or about 1000 mg of a non-racemic mixture of (R)-MDMA or pharmaceutically acceptable salt thereof or (S)-MDMA or pharmaceutically acceptable salt thereof, including all values and ranges there between. In embodiments, the present disclosure provides a method of treating a condition or disorder in a patient in need thereof (such as PTSD), the method comprising administering to the patient about 75 mg to about 1000 mg, about 75 mg to about 225 mg, about 500 mg to about 600 mg, or about 225 mg to about 600 mg of a non-racemic mixture of (R)-MDMA or pharmaceutically acceptable salt thereof or (S)-MDMA or pharmaceutically acceptable salt thereof.

In embodiments, the present disclosure provides a method of treating a condition or disorder in a patient in need thereof (such as PTSD), the method comprising administering to the patient 40 mg to 220 mg of (R)-MDMA or pharmaceutically acceptable salt thereof. In embodiments, the present disclosure provides a method of treating a condition or disorder in a patient in need thereof (such as PTSD), the method comprising administering to the patient about 20 mg, about 25 mg, about 30 mg, about 35 mg, about 40 mg, about 45 mg, about 50 mg, about 55mg, about 60 mg, about 65mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg or about 180 mg of a non-racemic mixture of (R)-MDMA or pharmaceutically acceptable salt thereof, including all values and ranges there between.

In embodiments, the present disclosure provides a method of treating a condition or disorder in a patient in need thereof (such as PTSD), the method comprising administering to the patient about 75 mg to 1000 mg of (R)-MDMA or pharmaceutically acceptable salt thereof. In embodiments, the present disclosure provides a method of treating a condition or disorder in a patient in need thereof (such as PTSD), the method comprising administering to the patient about 75 mg, about 85 mg, about 95 mg, about 105 mg, about 110 mg, about 115 mg, about 125 mg, about 135 mg, about 145 mg, about 155 mg, about 165 mg, about 175 mg, about 185 mg, about 195 mg, about 205 mg, about 215 mg, about 225 mg, about 235 mg, about 245 mg, about 255 mg, about 265 mg, about 275 mg, about 285 mg, about 295 mg, about 305 mg, about 315 mg, about 325 mg, about 335 mg, about 345 mg, about 355 mg, about 365 mg, about 375 mg, about 385 mg, about 395 mg, about 405 mg, about 415 mg, about 425 mg, about 435 mg, about 445 mg, about 455 mg, about 465 mg, about 475 mg, about 485 mg, about 495 mg, about 505 mg, about 515 mg, about 525 mg, about 535 mg, about 545 mg, about 555 mg, about 565 mg, about 575 mg, about 585 mg, about 595 mg, about 605 mg, about 615 mg, about 625 mg, about 635 mg, about 645 mg, about 655 mg, about 665 mg, about 675 mg, about 685 mg, about 695 mg, about 705 mg, about 715 mg, about 725 mg, about 735 mg, about 745 mg, about 755 mg, about 765 mg, about 775 mg, about 785 mg, about 795 mg, about 805 mg, about 815 mg, about 825 mg, about 835 mg, about 845 mg, about 855 mg, about 865 mg, about 875 mg, about 885 mg, about 895 mg, about 905 mg, about 915 mg, about 925 mg, about 935 mg, about 945 mg, about 955 mg, about 965 mg, about 975 mg, about 985 mg, about 995 mg, or about 1000 mg of R)-MDMA or pharmaceutically acceptable salt thereof, including all values and ranges there between. In embodiments, the present disclosure provides a method of treating a condition or disorder in a patient in need thereof (such as PTSD), the method comprising administering to the patient about 75 mg to about 1000 mg, about 75 mg to about 225 mg, about 500 mg to about 600 mg, or about 225 mg to about 600 mg of (R)-MDMA or pharmaceutically acceptable salt.

In embodiments of the methods described herein, the enantiomeric purity of the (R)-MDMA administered to the patient is ≥90%, ≥95%, ≥96%, ≥97%, ≥98%, ≥99%, ≥99.5%, ≥99.9%, or ≥99.99%. In embodiments of the methods described herein, the enantiomeric purity of the (R)-MDMA administered to the patient is ≥98%. In embodiments of the methods described herein, the enantiomeric purity of the (R)-MDMA administered to the patient is 100%. In embodiments of the methods described herein, the patient is administered (R)-MDMA hydrochloride (HCl).

According to the methods of the present disclosure, the compositions of the present disclosure may be administered to the patient in need thereof using any suitable route of administration known to those skilled in the art, including oral, parenteral (e.g., intravenous, subcutaneous, intradermal, intramuscular, intradermal, intrapleural, intracerebral, and intra-articular), topical (e.g., to both skin and mucosal surfaces, including airway surfaces, and transdermal administration), inhalation (e.g., via an aerosol), rectal, transmucosal, intranasal, buccal, sublingual, vaginal, intrathecal, intraocular, transdermal.

In embodiments, the composition of the present disclosure is orally administered.

### Numbered Embodiments of the Disclosure

In addition to the disclosure above, the Examples below, and the appended claims, the disclosure sets forth the following numbered embodiments.
1. A pharmaceutical composition comprising a non-racemic mixture of (R)-3,4-methylenedioxymethamphetamine (MDMA) or pharmaceutically acceptable salt thereof and (S)-MDMA or pharmaceutically acceptable salt thereof.
2. The pharmaceutical composition of embodiment 1, wherein the weight ratio of (R)-MDMA to (S)-MDMA is greater than 1.
3. The pharmaceutical composition of embodiment 1 or 2, capable of providing, upon administration of the composition to a patient in need thereof, a greater therapeutic index compared to administration of an equal dose (by weight) of racemic MDMA.
4. The pharmaceutical composition of any of embodiments 1-3, capable of providing, upon administration of the composition to a patient in need thereof, increased levels of neurohormones compared to administration of an equal dose (by weight) of substantially pure (R)-MDMA.
5. The pharmaceutical composition of embodiment 4, wherein said neurohormones include oxytocin, vasopressin, or prolactin.
6. The pharmaceutical composition of any of embodiments 1-5, capable of providing, upon the administration of the composition to a patient in need thereof, increased stimulation of post-synaptic 5-HT2A signaling compared to administration of an equal dose (by weight) of racemic MDMA.
7. The pharmaceutical composition of any of embodiments 1-6, capable of providing, upon administration of the composition to a patient in need thereof, increased neurogenesis compared to administration of an equal dose (by weight) of racemic MDMA.
8. The pharmaceutical composition of any of embodiments 1-7, wherein the weight ratio of (R)-MDMA to (S)-MDMA is from 51:49 to 99:1.
9. The pharmaceutical composition of any of embodiments 1-8, wherein the weight ratio of (R)-MDMA to (S)-MDMA is 51:49, 52:48, 53:47, 54:46, 55:45, 56:44, 57:43, 58:42, 59:41, 60:40, 61:39, 62:38, 63:37, 64:36 , 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 or 99:1.
10. The pharmaceutical composition of any of embodiments 1-9, wherein the weight ratio of (R)-MDMA to (S)-MDMA is from 90:10 to 99:1.
11. The pharmaceutical composition of any of embodiments 1-10, wherein the weight ratio of (R)-MDMA to (S)-MDMA is 90:10, 91:9, 92:8, 93:7, 94:6, 95:5, 96:4, 97:3, 98:2 or 99:1.
12. The pharmaceutical composition of any of embodiments 1-11, wherein the weight ratio of (R)-MDMA to (S)-MDMA is greater than 90:10.

13. The pharmaceutical composition of any of embodiments 1-12, wherein the weight ratio of (R)-MDMA to (S)-MDMA is less than 1.
14. A pharmaceutical composition comprising a non-racemic mixture of a (R)-MDMA metabolite and a (S)-MDMA metabolite, or pharmaceutically acceptable salt thereof.
15. The pharmaceutical composition of embodiment 14, wherein the MDMA metabolite is 3,4-methylenedioxyamphetamine (MDA), 4-hydroxy-3-methoxymethamphetamine (HMMA), 4-hydroxy-3-methoxyamphetamine (HMA), 3,4-dihydroxyamphetamine (DHA), 3,4-methylenedioxyphenylacetone (MDP2P) or 3,4-methylenedioxy-N-hydroxyamphetamine.
16. The pharmaceutical composition of embodiment 14 or 15, wherein the MDMA metabolite is MDA.
17. The pharmaceutical composition of embodiment 16, wherein the weight ratio of (R)-MDA to (S)-MDA is greater than 1.
18. The pharmaceutical composition of any of embodiments 14-17, capable of providing, upon administration of the composition to a patient in need thereof, a greater therapeutic index compared to administration of an equal dose (by weight) of racemic MDA.
19. The pharmaceutical composition of any of embodiments 14-18, capable of providing, upon administration of the composition to a patient in need thereof, increased levels of neurohormones compared to administration of an equal dose (by weight) of substantially pure (R)-MDMA.
20. The pharmaceutical composition of embodiment 19, wherein said neurohormones are oxytocin, vasopressin, or prolactin.
21. The pharmaceutical composition of any of embodiments 14-20, capable of providing, upon administration of the composition to a patient in need thereof, increased stimulation of post-synaptic 5-HT2A signaling compared to administration of an equal dose (by weight) of racemic MDA.
22. The pharmaceutical composition of any of embodiments 14-21, capable of providing, upon administration of the composition to a patient in need thereof, increased neurogenesis compared to administration of an equal dose (by weight) of racemic MDA.
23. The pharmaceutical composition of any of embodiments 16-22, wherein the weight ratio of (R)-MDA to (S)-MDA is from 51:49 to 99:1.
24. The pharmaceutical composition of any of embodiments 16-23, wherein the weight ratio of (R)-MDA to (S)-MDA is 51:49, 52:48, 53:47, 54:46, 55:45, 56:44, 57:43, 58:42, 59:41, 60:40, 61:39, 62:38, 63:37, 64:36 , 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 or 99:1.
25. The pharmaceutical composition of any of embodiments 16-24 wherein the weight ratio of (R)-MDA to (S)-MDA is from 90:10 to 99:1.
26. The pharmaceutical composition of any of embodiments 16-25, wherein the weight ratio of (R)-MDA to (S)-MDA is 90:10, 91:9, 92:8, 93:7, 94:6, 95:5, 96:4, 97:3, 98:2 or 99:1.
27. The pharmaceutical composition of embodiments 16-26, wherein the weight ratio of (R)-MDA to (S)-MDA is greater than 90:10.
28. The pharmaceutical composition of any of embodiments 1-27, wherein the composition is an oral dosage form.
29. The pharmaceutical composition of embodiment 28, wherein the oral dosage form is a tablet or capsule.
30. A method of reducing the incidence of adverse events associated with the administration of racemic MDMA, the method comprising administering a therapeutically effective amount of the composition of any one of embodiments 1-29 to a patient in need thereof.
31. The method of embodiment 30, wherein the adverse event associated with the administration of racemic MDMA is hypertension.
32. The method of embodiment 30, wherein the adverse event associated with the administration of racemic MDMA is stroke.
33. The method of embodiment 30, comprising treating the patient for symptoms of post-traumatic stress disorder (PTSD).
34. The method of embodiment 30, comprising treating the patient for symptoms of generalized anxiety disorder.
35. The method of embodiment 30, comprising treating the patient for an eating disorder.
36. The method of embodiment 30, comprising selecting the administered ratio of (R)-MDMA to (S)-MDMA, or a metabolite thereof, and dose of the composition to reduce the incidence of adverse events in obese patients.
37. The method of embodiment 30, comprising selecting the administered ratio of (R)-MDMA to (S)-MDMA, or a metabolite thereof, and dose of the composition to reduce the incidence of adverse events in hypertensive patients.

### EXAMPLES

The present invention is further illustrated by reference to the following Examples. However, it is noted that that Examples, like the embodiments described above, are illustrative and are not to be construed as restricting the scope of the invention in any way.

### Example 1

Primary rat cortical neuron experimental methods: Female Wistar rats of 17 days gestation were killed by cervical dislocation and the fetuses (typically 6 to 8 in number) were removed from the uterus. Fetal brains were placed in ice-cold medium of Leibovitz (L15, Gibco, France). Cortex was dissected and meninges were carefully removed. The cortical neurons were dissociated by trypsinization (trypsin-EDTA, Gibco) in the presence of DNAse I (Roche, France). The reaction was stopped by addition of Dulbecco's Modified Eagle Medium (DMEM; Gibco) with 10% of fetal bovine serum (FBS; Gibco). The suspension was triturated with a 10-ml pipette and a 21-gauge needle syringe and centrifuged. The pellet of dissociated cells was resuspended in a medium consisting of Neurobasal (Gibco) supplemented with 2% B27 supplement (Gibco), 0.5 mM L-Glutamine (Gibco), and an antibiotic-antimycotic mixture. Viable cells were counted in a Neubauer cytometer and cells were seeded in 96-well plates (Costar) precoated with poly-L-lysine at 10,000 cells/well.

Compounds, including negative control (vehicle), positive control (Donepezil 250 nM), and test article(s) at one or more concentrations, were added to the cultures on the plating day (Day0). Stock solutions were prepared in sterile water at 10 mM, and stored at −20° C. until use. Further dilution will be performed in culture in the medium on the day of the treatment. 0.1% Sterile water will be present in all tested conditions. The following conditions were tested: 1) Racemic MDMA (10000, 1000, 100, 10, 1, 0.1, 0.01, 0.001 nM), 2) S(+)-MDMA (10000, 1000, 100, 10, 1, 0.1, 0.01, 0.001 nM), 3) R(−)-MDMA (10000, 1000, 100, 10, 1, 0.1, 0.01, 0.001 nM), 4) Racemic MDA (10000, 1000, 100, 10, 1, 0.1, 0.01, 0.001 nM), 5) S(+)-MDA (10000, 1000, 100, 10, 1, 0.1, 0.01, 0.001 nM), and 6) R(−)-MDA (10000, 1000, 100, 10, 1, 0.1, 0.01, 0.001 nM).

The experimental protocol was performed in 2 independent cultures (i.e., from 2 different pregnant rats). For each culture, each condition was tested in sextuplet (6 wells per condition per culture for a total of 12 wells per condition). Each plate contained 3 types of experimental conditions: the negative control condition treated with vehicle (0.1% sterile water), the positive control condition treated with Donepezil (250 nM) and test article conditions. After three days of plating and compound treatment (Day3), cultures were fixed with paraformaldehyde in phosphate buffered saline at 4° C. (PBS, 4%, Sigma). Then, all subsequent steps were performed at room temperature.

Cells were successively permeabilized for 30 minutes using 0.1% triton, saturated with PBS containing 3% bovine serum albumin (BSA) and incubated for 1 hour with anti-beta III tubulin antibody (T5168, Sigma). Cells were first washed 3 times and then were incubated for 1 hour with goat anti-mouse secondary antibody coupled with Alexa Fluor 488 (AF488, Invitrogen A11001). Finally, nuclei were stained with 4′-6-diamidino-2-phenylindole (DAPI). After rinsing the cells with PBS, the plate was imaged and neurite networks were examined and analyzed using a High-Content Screening platform (CellInsight CX5, Thermo Scientific) with integrated Photometrics high-resolution fluorescent camera, Olympus objective (10×) and HCS Studio Cell Analysis Software.

Approximately 2,000 neurons per well were analyzed using a validated cortex neuron outgrowth algorithm that utilizes parameters optimized for analysis of embryonic rat cortical neuron cultures. The HCS Studio Cell Analysis Software output will include values for neurite number total count, neurite total length (μm) and total number of branch points for each of the 6 wells per culture. The evaluation of neurite outgrowth was performed using the average number of neurites per neuron, the average total neurite length per neuron (μm) and the average number of branch points per neuron across 12 wells. Data also were transformed to percent (%) of the average vehicle control value. Results were expressed as mean (±s.e.m.) of the transformed (% of vehicle control) data. Statistical analyses were performed on transformed percent of the average vehicle control data for each measure for each test article using one-way analysis of variance (Anova, StatView or GraphPad Prism). Where applicable, Dunnett's test was used for multiple pairwise comparisons to the negative control (vehicle) condition. Transformed percent of the average vehicle control data for each measure for the positive control Donepezil (250 nM) was compared to the negative control (vehicle) condition using an unpaired t test. The level of significance was set at p value less than or equal to 0.05 (Anova (*) or t test (#), p<0.05).

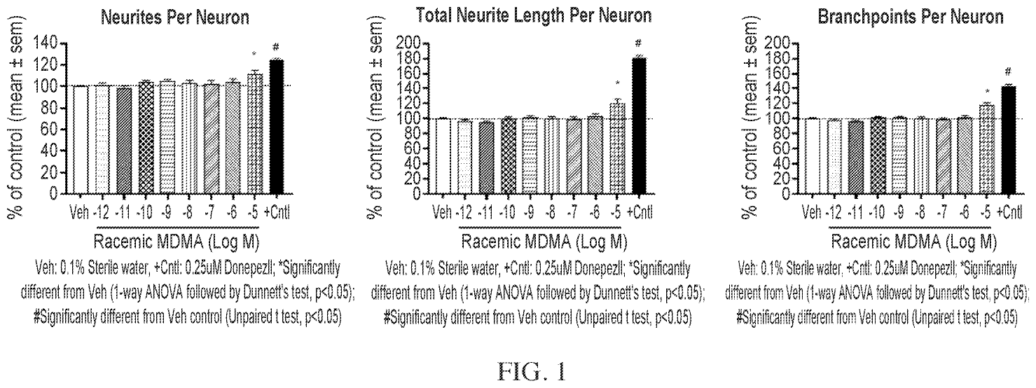

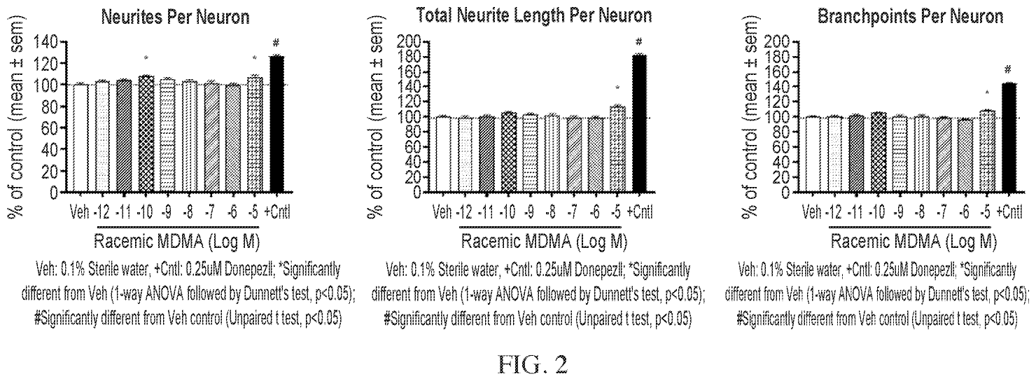

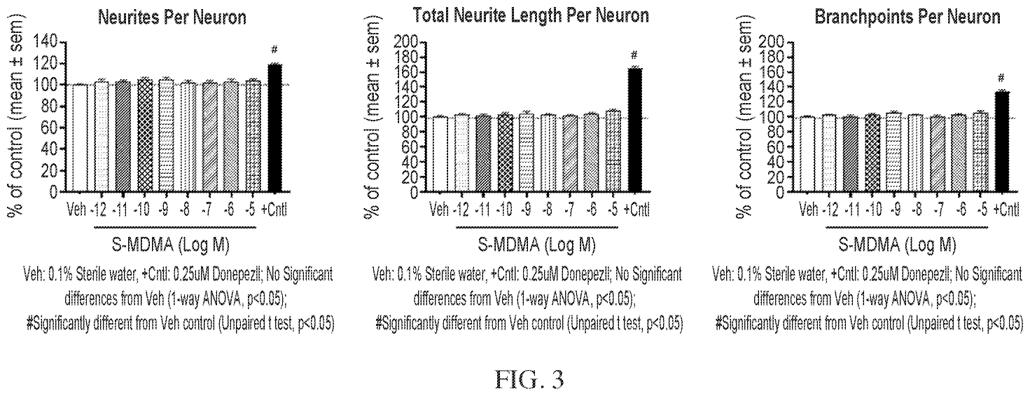

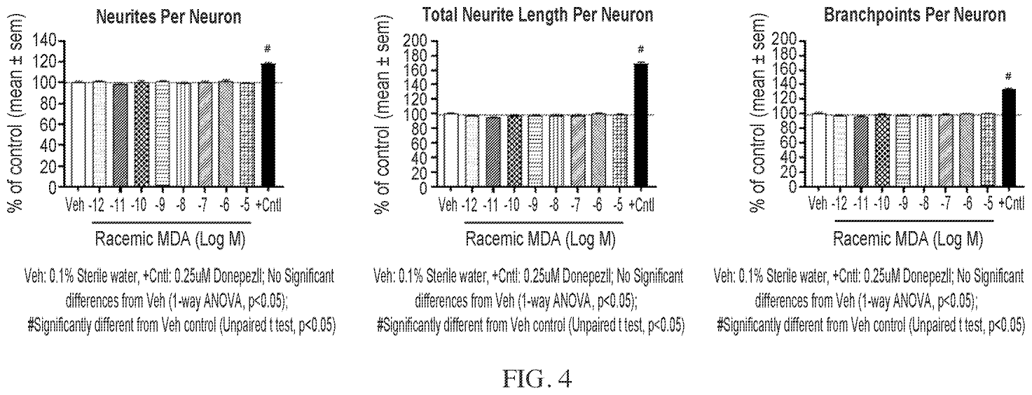

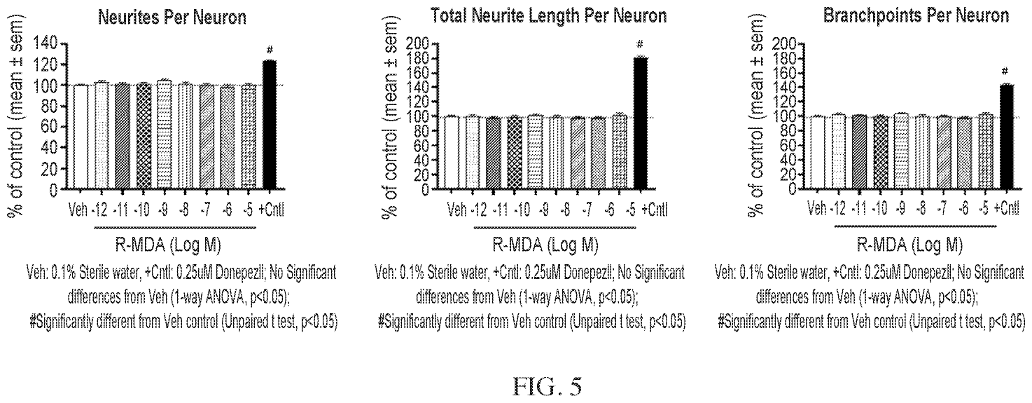

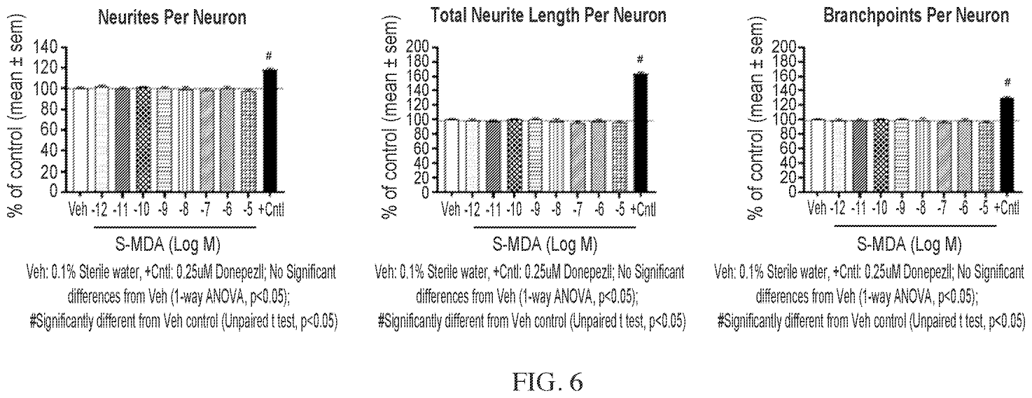

Experimental results: Under the conditions tested, in each of the 6 test article experiments, the positive control Donepezil (250 nM) demonstrated the expected effects of significantly increasing neurite number, neurite total length (μm) and total number of branch points compared to the 0.1% sterile water vehicle control. Racemic R,S(+/−)-MDMA significantly increased neurite number, neurite total length (μm) and total number of branch points at 10 μM compared to the 0.1% sterile water vehicle control (FIG. 1). R(−)-MDMA significantly increased neurite number, neurite total length (μm) and total number of branch points at 10 μM, and also significantly increased neurite number at 0.0001 μM, compared to the 0.1% sterile water vehicle control (FIG. 2). S(+)-MDMA, racemic R,S(+/−)-MDA, R(−)-MDA and S(+)-MDA did not significantly alter neurite number, neurite total length (μm), or total number of branch points at any tested concentration compared to the 0.1% sterile water vehicle control (FIGS. 3, 4, 5 and 6, respectively).

Summary: Of the 6 compounds tested, racemic R,S(+/−)-MDMA and R-MDMA significantly increased neurite outgrowth parameters under the conditions evaluated. Racemic and R-MDMA increased all three parameters measured at 10 μM. In contrast, S-MDMA, and racemic, R- and S-MDA, did not induce a statistically significant increase in any of the neurite parameters measured. These data suggest that racemic MDMA and R-MDMA may exhibit structural neuroplasticity, which may have therapeutic benefit in the treatment of stress-related disorders.

### Example 2

Primary rat cortical neuron experimental methods: Female Wistar rats of 17 days gestation are killed by cervical dislocation and the fetuses (typically 6 to 8 in number) are removed from the uterus. Their brains are placed in ice-cold medium of Leibovitz (L15, Gibco, France). Cortex is dissected and meninges are carefully removed. The cortical neurons are dissociated by trypsinization (trypsin-EDTA, Gibco) in presence of DNAse I (Roche, France). The reaction is stopped by addition of Dulbecco's Modified Eagle Medium (DMEM; Gibco) with 10% of fetal bovine serum (FBS; Gibco). The suspension is triturated with a 10-ml pipette and using a needle syringe 21G and centrifuged. The pellet of dissociated cells is resuspended in a medium consisting of Neurobasal (Gibco) supplemented with 2% B27 supplement (Gibco), 0.5 mM L-Glutamine (Gibco), an antibiotic-antimycotic mixture. Viable cells are counted in a Neubauer cytometer and cells are seeded in 96-well plate (Costar) precoated with poly-L-lysine at 10 000cells/well.

Each compound stock solution is prepared in sterile water at a concentration 1000 times higher compared to the highest tested concentration (i.e., 10 mM). Each stock solution is prepared separately to minimize the possibility of cross contamination between each isomeric form of the base molecule.

The protocol is performed in either 1 or 2 independent cultures (i.e., from 1 or 2 different pregnant rats). For each culture, each condition is performed in sextuplet (6 wells per condition per culture from 1 rat, or a total of 12 wells per condition when 2 rats/cultures are used). Each culture or plate contains 3 types of experimental conditions: the negative control condition treated with vehicle (0.1% sterile water), the positive control condition treated with Donepezil (250 nM), and the 8 test compound conditions treated with varied concentrations of MDMA derivative compounds.

Test compound at different concentrations or Donepezil are added to the cultures on the plating day (Day0). Cells are treated with the test compounds R(−)-MDMA and S(+)-MDMA as described in the following Tables below. For this study, 6 culture wells (1 rat) were treated with non-racemic mixtures containing enantiomeric ratios of S(+) ≥R(−) (Table 2), and 12 culture wells (2 rats) were treated with non-racemic mixtures containing enantiomeric ratios of R(−) ≥S(+) (Table 1).

TABLE 1
Test concentration ratios with R(−)-MDMA equivalent to or greater
than S(+)-MDMA in racemic and non-racemic mixtures

| R-MDMA (uM) | S-MDMA (uM) | % R -MDMA |
|---|---|---|
| 10 | 10 | 50.00 |
| 10 | 3 | 76.92 |
| 10 | 1 | 90.91 |
| 10 | 0.3 | 97.09 |
| 10 | 0.1 | 99.01 |
| 10 | 0.03 | 99.70 |
| 10 | 0.01 | 99.90 |
| 10 | 0 | 100.00 |

TABLE 2
Test concentration ratios with S(+)-MDMA equivalent to or greater
than R(−)-MDMA in racemic and non-racemic mixtures

| R-MDMA (uM) | S-MDMA (uM) | % S-MDMA |
|---|---|---|
| 10 | 10 | 50.00 |
| 3 | 10 | 76.92 |
| 1 | 10 | 90.91 |
| 0.3 | 10 | 97.09 |
| 0.1 | 10 | 99.01 |
| 0.03 | 10 | 99.70 |
| 0.01 | 10 | 99.90 |
| 0 | 10 | 100.00 |

After three days of plating and test compound treatment (Day3), cultures are fixed with paraformaldehyde in phosphate buffered saline at 4° C. (PBS, 4%, Sigma). Then all steps will be performed at room temperature. Cells are successively permeabilized 30 min using 0.1% triton, saturated with PBS containing 3% bovine serum albumin (BSA) and incubated 1 h with anti-beta III tubulin antibody (T5168, Sigma). Cells are first washed 3 times and are then incubated 1 h with goat anti-mouse secondary antibody coupled with Alexa Fluor 488 (AF488, Invitrogen A11001). Finally, nuclei are stained with 4′-6-diamidino-2-phenylindole (DAPI). After rinsing the cells with PBS, the plate is filmed and neurite networks are examined and analyzed using a High-Content Screening platform (CellInsight CX5, Thermo Scientific) with integrated Photometrics high-resolution fluorescent camera, Olympus objective (10×) and HCS Studio Cell Analysis Software.

Approximately 2000 neurons per well were analyzed using a validated Cortex Neuron Outgrowth algorithm established by Neurofit that utilizes parameters optimized for analysis of embryonic rat cortical neuron cultures. The HCS Studio Cell Analysis Software output will include values for neurite total count number, neurite total length (μm) and total number of branch points for each of the 6 wells per culture. The evaluation of neurite outgrowth was performed using the average number of neurites per neuron, the average of total neurite length per neuron (μm) and the average number of branchpoint per neuron across 6 wells of each culture (6 wells total (1 rat used) or 12 wells total (2 rats used). Data were transformed to % of the average vehicle control value.

Statistical data analysis: Results are expressed as mean (±s.e.m.) of the transformed (% of vehicle control) data. Statistical analysis of the data was performed using one way analysis of variance (Anova, StatView or GraphPad Prism). Where applicable, Dunnet's test was used for multiple pairwise comparisons to the negative control (vehicle) condition. Transformed percent of the average vehicle control data for each measure for the positive control Donepezil (250 nM) was compared to the negative control (vehicle) condition using an unpaired t test. The level of significance was set at p value less than or equal to 0.05 (Anova (*) or t test (#), p<0.05).

Experimental results: The positive control Donepezil (250 nM) demonstrated the expected effects of significantly increasing neurite number, neurite total length (um) and total number of branchpoints compared to the vehicle control for all of the conditions tested, with the exception of one sample in which the increase in the average number of neurites did not reach statistical significance (10 uM S(+), variable R(−) treatment conditions) (FIG. 1 and FIG. 2).

Given that a significant increase for this same neurite parameter was observed with the positive control in a parallel treatment group (10 uM R(−), variable S(+) conditions) (neuron culture derived from same rat), this non-significant neurite parameter effect of the positive control may be due to a technical issue with that particular test sample (FIG. 1 and FIG. 2, left panels). The racemic mixture conditions with 10 uM R(−) and 10 uM S(+) showed significant inducement of neurite outgrowth for all three parameters tested (FIG. 1 and FIG. 2). The non-racemic mixture with enantiomeric ratio of 10 uM R(−):3 uM S(+) showed significant inducement of neurite outgrowth across all three parameters tested, all enantiomeric ratios tested except for 10 uM R(−):0.3 uM S(+) significantly increased the average total neurite length per neuron, and the enantiomeric ratio mixtures of 10 uM R(−):0.01, 0.1, 1, or 3 uM S(+) significantly increased the average number of branchpoints per neuron (FIG. 1). The non-racemic mixture with enantiomeric ratio of 10 uM S(+):3 uM R(−) significantly increased total neurite length per neuron, and was the only condition to significantly affect any of the neurite parameters tested with S(+) >R(−) ratios (FIG. 2). R(−)-MDMA alone (10 uM) significantly induced the parameter of total neurite length per neuron, and S(+)-MDMA (10 uM) alone did not significantly induce any of the three tested neurite parameters (FIG. 1 and FIG. 2).

Summary: Racemic mixtures of R(−)- and S(+)-MDMA (10 uM) significantly increased all three neurite parameters, consistent with the effects of racemic MDMA observed in the experiment represented in FIG. 1. Non-racemic mixtures of R(−)- and S(+)-MDMA at enantiomeric ratio of 10 uM:3 uM, respectively, significantly increased all three neurite parameters, including the two in which 10 uM R(−) alone did not have significant effects in this investigation (number of neurites per neuron and number of branchpoints per neuron). These data suggest that while the S(+) enantiomer does not have neuritogenesis inducing properties alone (10 uM), it can contribute to the inducement of neuritogenesis when in racemic and non-racemic mixtures with the R(−) enantiomer. These observations of R(−) and S(+) enantiomers in non-racemic mixtures synergistically promoting neuritogenesis as a form of structural neuroplasticity may have therapeutic utility in the treatment of stress-related disorders.

While the present invention is capable of being embodied in various forms, the description above of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. An oral dosage form comprising:
(i) (R)-3,4-methylenedioxymethamphetamine ((R)-MDMA) or pharmaceutically acceptable salt thereof in an enantiomeric purity of greater than 98%; and
(ii) a pharmaceutically acceptable carrier.

2. The oral dosage form of claim 1, wherein the salt is a hydrochloride salt.

3. The oral dosage form of claim 1, wherein the dosage form comprises 75 to 1000 mg of (R)-MDMA.

4. The oral dosage form of claim 3, wherein the dosage form comprises 75 to 225 mg of (R)-MDMA.

5. The oral dosage form of claim 3, wherein the dosage form comprises 225 to 600 mg of (R)-MDMA.

6. The oral dosage form of claim 5, wherein the dosage form comprises 500 to 600 mg of (R)-MDMA.

7. The oral dosage form of claim 5, wherein the dosage form comprises 300 to 600 mg of (R)-MDMA.

8. The oral dosage form of claim 1, wherein the enantiomeric purity of (R)-MDMA is greater than 99%.

9. The oral dosage form of claim 8, wherein the salt is a hydrochloride salt.

10. The oral dosage form of claim 1, wherein the dosage form comprises
300 to 600 mg of (R)-MDMA, and
wherein the (R)-MDMA is a hydrochloride salt.

11. The oral dosage form of claim 10, wherein the dosage form comprises 500 to 600 mg of (R)-MDMA.

12. The oral dosage form of claim 11, wherein the enantiomeric purity of (R)-MDMA is greater than 99%.

13. The oral dosage form of claim 12, wherein the enantiomeric purity of (R)-MDMA is 100%.

14. A method of treating post-traumatic stress disorder in a patient in need thereof, the method comprising orally administering to the patient an oral dosage form, the oral dosage form comprising:
(i) (R)-MDMA or pharmaceutically acceptable salt thereof in an enantiomeric purity of greater than 98%; and
(ii) a pharmaceutically acceptable carrier.

15. The method of claim 14, wherein the salt is a hydrochloride salt.

16. The method of claim 14, wherein 75 to 1000 mg of (R)-MDMA is administered to the patient.

17. The method of claim 16, wherein 75 to 225 mg of (R)-MDMA is administered to the patient.

18. The method of claim 16, wherein 225 to 600 mg of (R)-MDMA is administered to the patient.

19. The method of claim 18, wherein 500 to 600 mg of (R)-MDMA is administered to the patient.

20. The method of claim 14, wherein the 300 to 600 mg of (R)-MDMA is administered to the patient.

21. The method of claim 14, wherein the enantiomeric purity of (R)-MDMA is greater than 99%.

22. The method of claim 21, wherein the salt is a hydrochloride salt.

23. A method of treating post-traumatic stress disorder in a patient in need thereof, the method comprising orally administering to the patient 300 to 600 mg of (R)-MDMA in an enantiomeric purity of greater than 98%, wherein the (R)-MDMA is a hydrochloride salt.

24. The method of claim 23, wherein 500 to 600 mg of (R)-MDMA is administered.

25. The method of claim 24, wherein the enantiomeric purity of (R)-MDMA is greater than 99%.

26. The method of claim 25, wherein the enantiomeric purity of (R)-MDMA is 100%.

27. A method of treating an eating disorder in a patient in need thereof, the method comprising orally administering to the patient an oral dosage form, the oral dosage form comprising:
(i) (R)-MDMA or pharmaceutically acceptable salt thereof in an enantiomeric purity of greater than 98%; and
(ii) a pharmaceutically acceptable carrier.
